(19) United States
(12) Patent Application Publication
(10) Pub. No.: US 2024/0285976 A1
(43) Pub. Date: Aug. 29, 2024

(54) ULTRA-HIGH X-RAY PHOTON RADIATION FOR FLASH RADIOTHERAPY

(71) Applicant: AcceleRAD Technologies, Inc., Hayward, CA (US)

(72) Inventor: Curtis Allen, Redwood City, CA (US)

(21) Appl. No.: 18/587,377

(22) Filed: Feb. 26, 2024

Related U.S. Application Data
(60) Provisional application No. 63/448,203, filed on Feb. 24, 2023.

(51) Int. Cl. A61N 5/10; H01J 35/14

(52) U.S. Cl. CPC A61N 5/1077; H01J 35/153

(57) ABSTRACT

Vacuum electron devices (VEDs) include an electron accelerator that generates an electron beam from an electron source along a central axis. Targets are arranged on a circular arc and generate photons upon impact by the electron beam. A controllable magnetic field generator generates a magnetic field to change a trajectory of the electron beam, including a first field region that causes the electron beam to diverge from the central axis and a second field region that causes the electron beam to converge toward the central axis to impact a selected target.

### BACKGROUND OF THE INVENTION

The present invention relates to radiotherapy and, more particularly, systems and methods for administering radiotherapy that change an electron beam path and corresponding radiation direction.

Radiotherapy makes use of high-energy electromagnetic radiation, such as X-rays (photons), to treat a patient by selectively damaging harmful tissue, such as cancerous tumors. However, such therapies can cause adverse effects to healthy tissue surrounding the target, which limits the dose that can be safely applied. This can limit the efficacy of the radiotherapy and its ability to completely destroy a tumor.

### BRIEF SUMMARY OF THE INVENTION

A vacuum electron device (VED) includes an electron accelerator that generates an electron beam from an electron source along a central axis. Targets are arranged on a circular arc and generate photons upon impact by the electron beam. A controllable magnetic field generator generates a magnetic field to change a trajectory of the electron beam, including a first field region that causes the electron beam to diverge from the central axis and a second field region that causes the electron beam to converge toward the central axis to impact a selected target.

A VED includes an electron accelerator that generates an electron beam from an electron source along a first axis. A redirecting magnetic field redirects the electron beam from the first axis to a second axis. Targets are arranged on a circular arc and generate photons upon impact by the electron beam. A controllable magnetic field generator generates a magnetic field to change a trajectory of the electron beam, including a first field region that causes the electron beam to diverge from the second axis and a second field region that causes the electron beam to converge toward the second axis to impact a selected target.

A VED includes a first electron accelerator that generates a first electron beam from a first electron source along a central axis in a first direction. A second electron accelerator generates a second electron beam from a second electron source along the central axis in a second direction, opposite to the first direction. A first set of targets are arranged on a first circular arc and generate photons upon impact by the first electron beam. A second set of targets are arranged on a second circular arc and generate photons upon impact by the second electron beam. A first controllable magnetic field generator generates a first magnetic field to change a trajectory of the first electron beam, including a first diverging region that causes the first electron beam to diverge from the central axis and a first converging field region that causes the first electron beam to converge toward the central axis to impact a selected first target. A second controllable magnetic field generator generates a second magnetic field to change a trajectory of the second electron beam, including a second diverging region that causes the second electron beam to diverge from the central axis and a second converging field region that causes the second electron beam to converge toward the central axis to impact a selected second target. Photons generated by the first target and photons generated by the second target intersect a same focal region.

These and other features and advantages will become apparent from the following detailed description of illustrative embodiments thereof, which is to be read in connection with the accompanying drawings.

### BRIEF DESCRIPTION OF THE DRAWINGS

The disclosure will provide details in the following description of preferred embodiments with reference to the following figures wherein:

FIG. 1 is diagram of a linear electron accelerator, in accordance with an embodiment of the present invention;

FIG. 2 is a diagram of a radiotherapy apparatus that has a target assembly on a circular arc, with magnetic fields guiding an electron beam to the target assembly, in accordance with an embodiment of the present invention;

FIG. 2A is a diagram of a radiotherapy apparatus that further includes a collimator to shape a resulting beam of photons, in accordance with an embodiment of the present invention;

FIG. 3 is a diagram of a radiotherapy apparatus that has a target assembly on a circular arc in a horizontal plane, in accordance with an embodiment of the present invention;

FIG. 4 is a diagram of an electron beam path passing through a magnetic field, in accordance with an embodiment of the present invention;

FIG. 5 is a diagram of electron beam paths through a diverging vacuum chamber and a converging vacuum chamber, in accordance with an embodiment of the present invention;

FIG. 6 is a cross-sectional view of a target assembly having multiple individual targets, in accordance with an embodiment of the present invention;

FIG. 7 is a cross-sectional view of a target assembly being formed from a continuous piece of target material;

FIG. 8 is a diagram showing multiple beam pattern configurations, where the electron beams are guided to targets on the target assembly in different orders, in accordance with an embodiment of the present invention;

FIG. 9 is a diagram of a radiotherapy apparatus that includes a magnetic field to change an axis of operation, in accordance with an embodiment of the present invention;

FIG. 10 is a graph that shows a relationship between depth and dosage for beams of different energies, in accordance with an embodiment of the present invention;

FIG. 11 is a diagram of a radiotherapy apparatus that applies two electron beams at once, in accordance with an embodiment of the present invention;

FIG. 12 is a graph that shows a relationship between depth and dosage for beams of different energies, particularly including the use of multiple beams, in accordance with an embodiment of the present invention;

FIG. 13 is a diagram of beam paths in a horizontal plane relative to a patient, in accordance with an embodiment of the present invention;

FIG. 14 is a diagram of a radiotherapy apparatus that applies two electron beams at once at an angle, in accordance with an embodiment of the present invention;

FIG. 15 is a block/flow diagram of a radiotherapy method that moves an electron beam between multiple targets, in accordance with an embodiment of the present invention; and

FIG. 16 is a block diagram of a controller that can perform beam control, in accordance with an embodiment of the present invention.

### DETAILED DESCRIPTION

The beam path trajectory of a radiotherapy apparatus can be changed using magnetic fields. This makes it possible to distribute the impact of the beam across multiple target locations and further to change the direction of the resulting radiation that reaches the patient. By distributing the location of the beam's impact on the target(s), the thermal effects of the beam are decreased, which extends the useful lifespan of the target(s). Additionally, by changing the direction of the radiation as it passes through the patient, an effective dose can be delivered to a selected region while decreasing the exposure of surrounding healthy tissue.

A vacuum electron device (VED), such as a linear particle accelerator, may use radio frequency (RF) fields to accelerate a pulsed electron beam to a megavolt velocity. Photons are generated when each pulse of the accelerated electron beam impacts a Bremsstrahlung converter, referred to herein as a target. The target may be made from a material having a high atomic number. When an electron strikes the target, it decelerates, releasing photons that continue in the same direction as the beam's direction. These photons make up the radiation that is used for radiotherapy.

However, the impact of the pulsed electron beam on the target also heats the target. For example, an electron beam can raise a target's material to over 1300° C. in a few microseconds and the high temperature caused by repeated pulses on a single target location, can cause failure in the target and can limit the number of electrons (current), and consequently the photon dose rate, that a given target can generate before failure.

The adverse effects of heat in the target can be mitigated by distributing pulsed electrons over many targets or target impact locations and this can be achieved using magnetic fields to deflect the accelerated electron beam off its original axis, scanning the beam from side-to-side or end-to-end along a given plane. Another magnetic field is then used to bring the deflected electron beam trajectory back toward its original axis, such that the beam paths are all directed toward a common focal point.

As the pulsed electron beam is scanned across the target(s), it temporarily impacts one location to produce a high dose rate (e.g., in excess of 40 Gy/sec). Between pulses, the deflection angle is electronically changed to redirect the beam to a next target location and the process is repeated. The electron beam is moved before the target experiences a thermal failure, and each target is positioned such that it has a striking surface substantially perpendicular to the axis of its electron beam's trajectory. The targets may be positioned in a circularly curved arc, with curvature that algins the axis of photon creation with a common focal point that is the same distance from all the target locations. The result is that the resulting photon beams, along their different respective trajectories, deliver the same dose rate to the focal point.

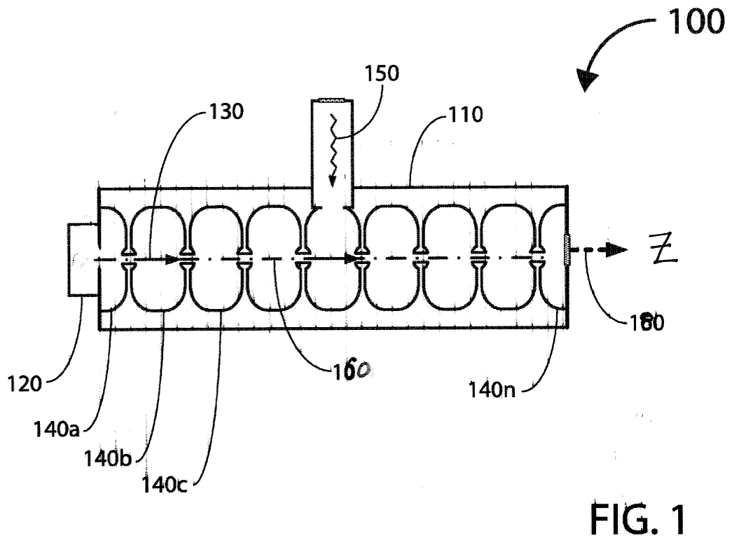

Referring now to FIG. 1, a schematic 100 of an exemplary linear electron accelerator 110 with an electron gun 120, and an RF accelerating structure 140. A pulsed electron beam 130 originates at the electron gun 120 along an axis 160 (referred to herein as the Z-axis), which is the common axis for both the RF accelerating structure and the electron gun 120. The pulsed electron beam 130 is accelerated through cavities 140a, 140b, 140c, . . . 140n, which are powered by microwave power 150, also known as RF power or electromagnetic power. The linear electron accelerator 110 thus produces a high-energy pulsed electron beam 180 (“e-”) as its output. The linear electron accelerator 110 and the electron gun 120 supply high energy electrons which are used to generate photons.

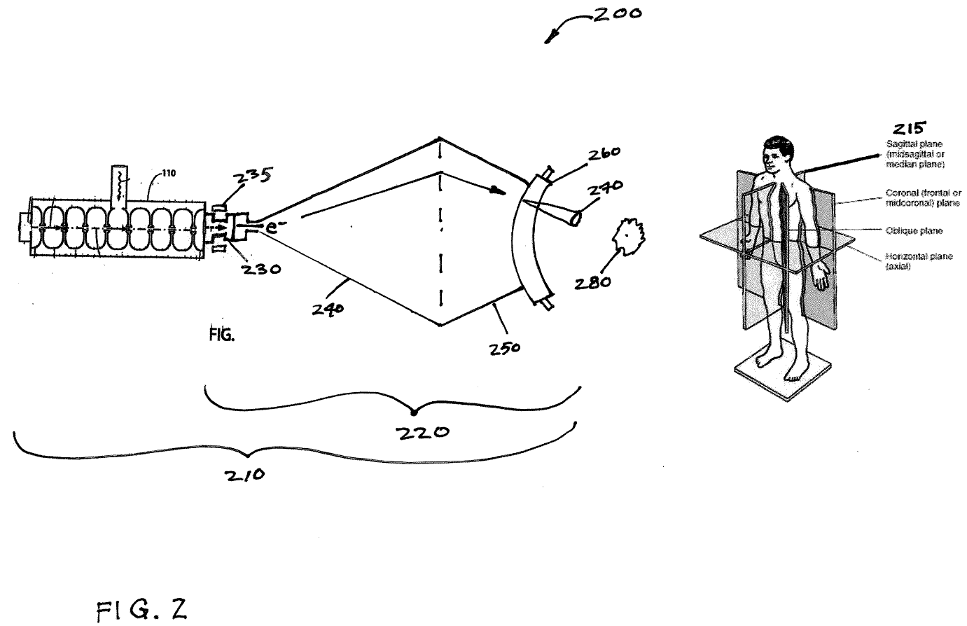

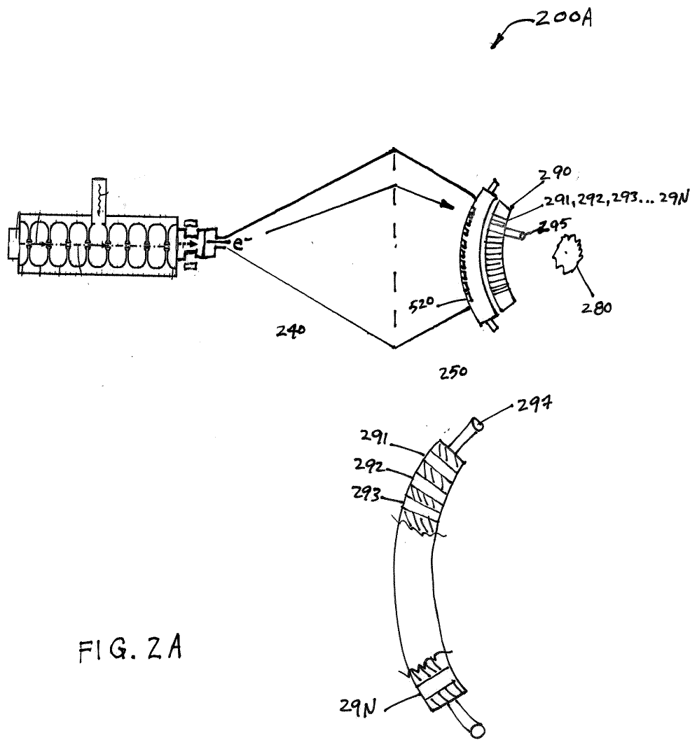

Referring now to FIG. 2, a schematic 200 is shown of an accelerator beam line 210, beginning with a linear electron accelerator 110 followed by a scan section 220, mounted vertically in the sagittal plane 215 and sharing the same high-vacuum envelope as the linear accelerator 110. The scan section 220 includes a drift tube 230, which can also incorporate one or more quadrupole magnets 235 to shape the pulse electron beam 180 that is output by the linear accelerator 110 and also to change the diameter of the pulsed electron beam 180. The scan section 220 may further include a diverging vacuum chamber 240 which deflects the electron beam's trajectory away from the original axis 160 and a converging vacuum chamber 250, where the deflected electron beam trajectories are redirected back toward the original axis 160, and a target assembly 260 that converts the high-energy pulsed electrons 180 to X-rays or photons 270. These X-rays or photons 270 may then be used to treat a tumor or lesion 280.

The distance between the target assembly 260 and the lesion 280 is the source-to-axis distance (SAD). Changing the SAD changes the dose that is delivered to the lesion 280, as radiation intensity diminishes according to the inverse square law. The angle of the converging electron beams may be adjusted by changing the strength of the magnetic field in the converging vacuum chamber 250, and the target assembly 260 may be designed to have an impact surface that is perpendicular to the converging electron beams for a particular SAD. The SAD may thus be changed by changing the strength of the magnetic field and by selecting an appropriate target assembly 260. The patient may then be positioned to place the lesion 280 at the appropriate location with a short SAD, thereby effectively increasing the delivered dose. For example, a SAD of 45 cm will see a dose rate about five times higher than would be delivered with a SAD of 100 cm.

The magnetic fields in the converging vacuum chamber 250 may be controlled by a controllable field generator. The controllable field generator may include one or more electromagnets, which generate magnetic fields in response to an applied current. This current may be controlled by an analog control or by digital control, such that the angle of the electron beams in the converging vacuum chamber 250 may be controlled by changing the current that is applied to the electromagnet. Different electron beam energies will furthermore need different magnetic field strengths. For example, a stronger magnetic field will be needed to apply the appropriate deflection to an electron beam with an energy of 12 MeV than would be needed to apply the same deflection to an electron beam with an energy of 10 MeV.

Referring now to FIG. 2A, a schematic 200A is shown that includes a collimator 290, shown in a cross-sectional exploded view, which may be made of a high atomic number metal. The collimator 290 may have respective apertures 291, 292, 293, . . . 29N for the targets 520. The apertures may have sizes ranging from about 4 mm to about 5 cm in diameter and may have any appropriate shape, such as cylindrical apertures, cone-shaped apertures that expand outward toward the lesion 280, and asymmetrical apertures that have a shape tailored to the lesion 280.

The collimator apertures may also be positioned on a circular arc, coaxial with the electron beam 180, and each have a central axis that is perpendicular to the striking surface of the targets 520. The circular arc of the collimator 290 may have a same center point as a circular arc of the targets 520. The collimator 290 may be used to remove and/or attenuate unwanted or off-axis photons, producing photon beams with cylindrical pencil beams of different sizes, expanding round or cone-shaped beams, or asymmetrical beam shapes 295 directed at the lesion 280. The collimator 290 absorbs photon radiation and may be cooled by convection or by a cooling medium, such as water. The path of the cooling media 297 may be routed around the apertures so as not to attenuate the photon beams.

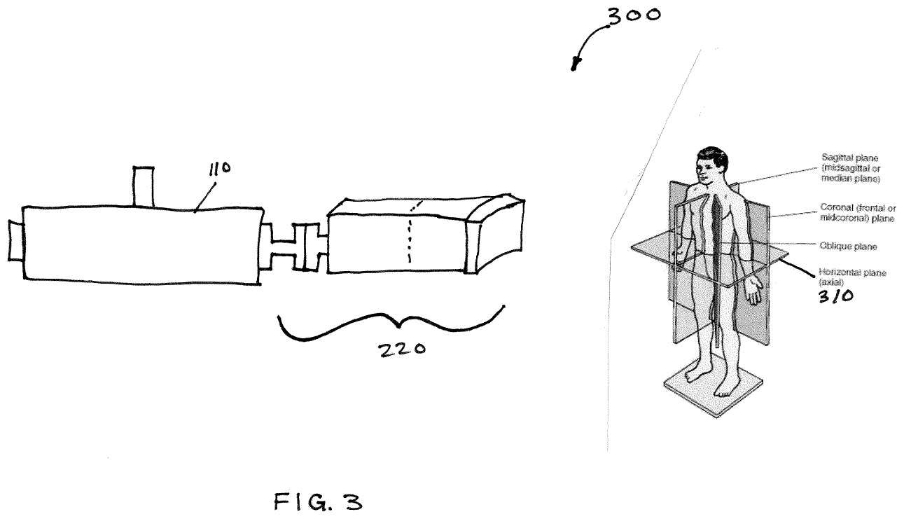

Referring now to FIG. 3, an alternate embodiment 300 of the schematic 200 is shown. In some embodiments, the scan section 220 may be mounted horizontally in the transverse or horizontal plane 310. In such embodiments, the target assembly 260 may be oriented such that the targets 520 lie along the horizontal plane 310, with the electron beam being scanned through this horizontal plane.

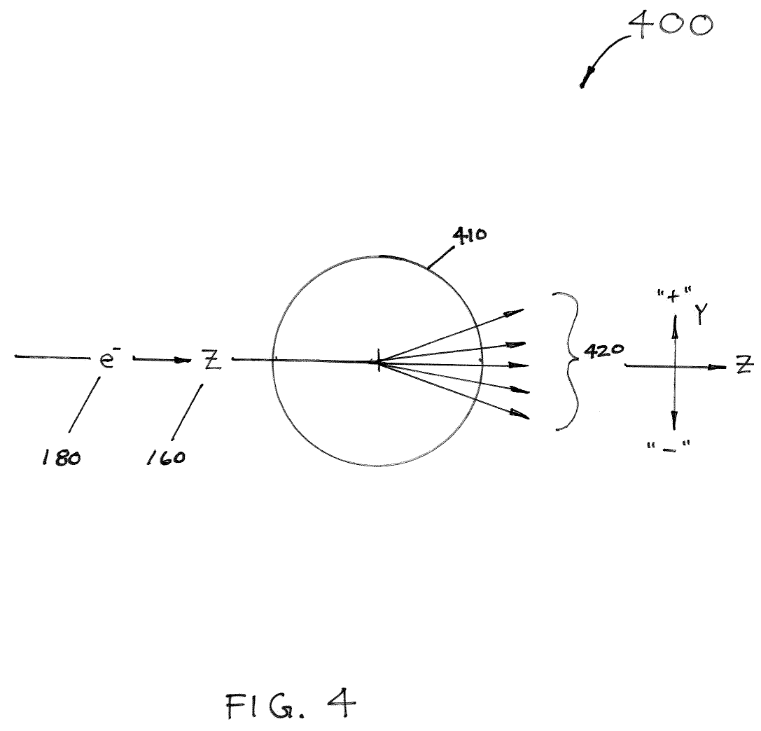

Referring now to FIG. 4, a diagram 400 of the pulsed electron beam 180 is shown being deflected outward, away from the original Z-axis 160, by one or more magnetic fields 410, and into diverging trajectories 420. The diverging trajectories 420 have increasing or differing angles in both an above “+” region and a below “−” region relative to the original Z-axis 160 along a Y-axis. The magnetic fields 410 may be generated by one or more controllable field generators, for example in the form of electromagnets, that are positioned around the Z-axis and that can be selectively activated to direct the beam toward particular targets.

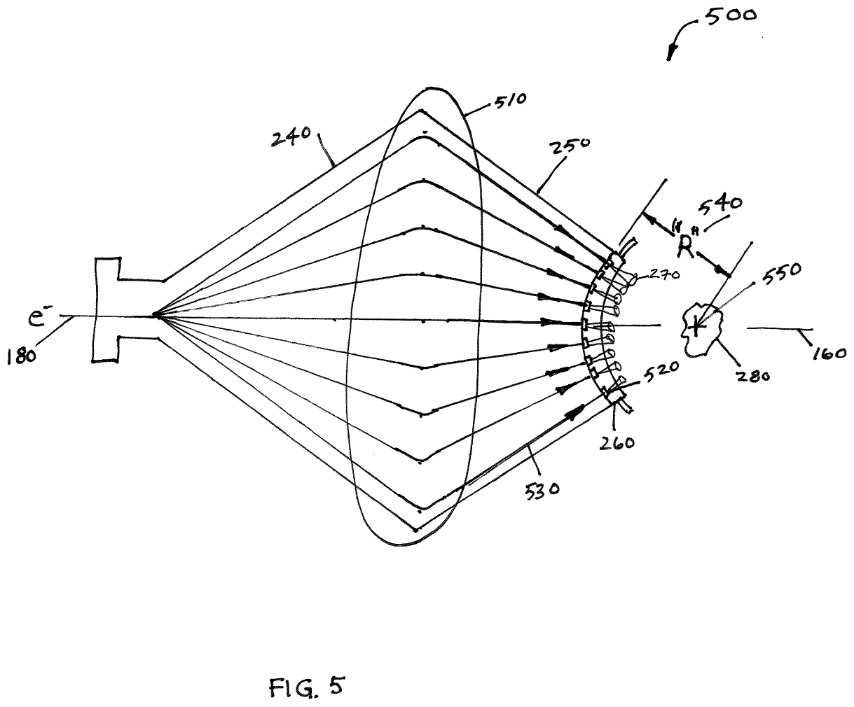

Referring now to FIG. 5, a cross-sectional view 500 is shown of a system that has no collimator 290. The pulsed electron beam 180 is deflected and travels through the diverging vacuum chamber 240 and is scanned from one end of the diverging pattern to the other. As the electron beam travels through the converging vacuum chamber 250, one or more magnetic fields 510 causes the electron beam 180 to converge back toward the original Z-axis 160. Thus the magnetic fields include a first region in the diverging vacuum chamber 240 and a second region in the converging vacuum chamber 250.

On the converging path, the electron beam 180 impacts one of the targets 520 arranged in a target assembly 260. Each converging beam trajectory 530 has a common focal point or focal point 550 within the lesion 280. The targets 520 convert the pulsed electron beam 180 into high-energy X-rays (photons) 270, having the same focal length 540 or radius (“R”) and a common focal point or focal point 550 within the lesion 280.

Targets 520 may be made from, e.g., tungsten or a tungsten alloy and may include a copper layer. For example, the targets 520 may be formed from pure tungsten, or a tungsten alloy, such as tungsten rhenium, with rhenium percentages of up to about 25%. The copper layer may be metallurgically bonded to the tungsten or tungsten alloy target, between the tungsten or tungsten alloy and a coolant. The use of rhenium in such an alloy increases the recrystallization temperature and offers better strain properties, but also decreases the thermal conductivity.

The tungsten or tungsten alloy target may have a thickness between about 0.8 mm and about 1.2 mm, while the copper layer may have a thickness of greater than about 1.0 mm. The electron beam energy may be optimized for the target thickness and design to keep the material of the target below its recrystallization temperature and to keep the copper layer below its softening temperature (e.g., about 500° C.).

The copper layer also creates X-rays (photons) and may be used to conduct heat to coolant for the target. The coolant may include water, but may be any other appropriate substance. The copper layer may be damaged by electron beams producing about 0.25 Gy/s, which is well below the flash radiotherapy dose rate levels. The dwell time of an electron beam may be about 4 μs and may be pulsed at a rate of about 250 Hz or higher as needed. The pulse width times the pulse repetition rate equals the duty factor, and higher duty factors allow the pulsed current to be decreased while maintaining the radiotherapy dose rate. Lower pulsed electron currents also allow the pulse width to increase until the maximum sustainable amount of power is met.

Other beam energies may be used as well, with operating parameters determined by radiotherapy dose rates, keeping the energy delivered to the target low enough to prevent thermal failures. Radiotherapy performed below 10 MV has the added benefit of not producing neutron radiation.

For a single 10 megavolt, 400 mA electron pulse of less than 1 μs in duration, a target may return to ambient temperature in about 5 ms, at which time the target can withstand another electron pulse. Power density also affects this, as the spot size determines the penumbra and, for radiotherapy, a spot size of less than 2.0 mm at full-width-at-half-max (FWHM) is desirable. A water cooling system may provide an average power density capacity of about 1 kW/cm². Different materials, sizes, beam energies, pulse widths, and cooling strategies for the targets will affect how long the target needs to cool off, and so will affect how many different beam paths and targets are needed to provide effective treatment without thermal damage to the target(s).

In some examples, the dosage that is delivered to healthy tissues and to diseased tissue may both be high. In such cases, it has been found that the ratio between the damage to the diseased tissue and damage to the healthy tissue increases substantially. In contrast, if a lower dosage is delivered to healthy tissue is lower, then this advantage disappears. Thus, in some embodiments the amount of dosage delivered to healthy tissue may be kept at a similar level to that delivered to the lesion 280.

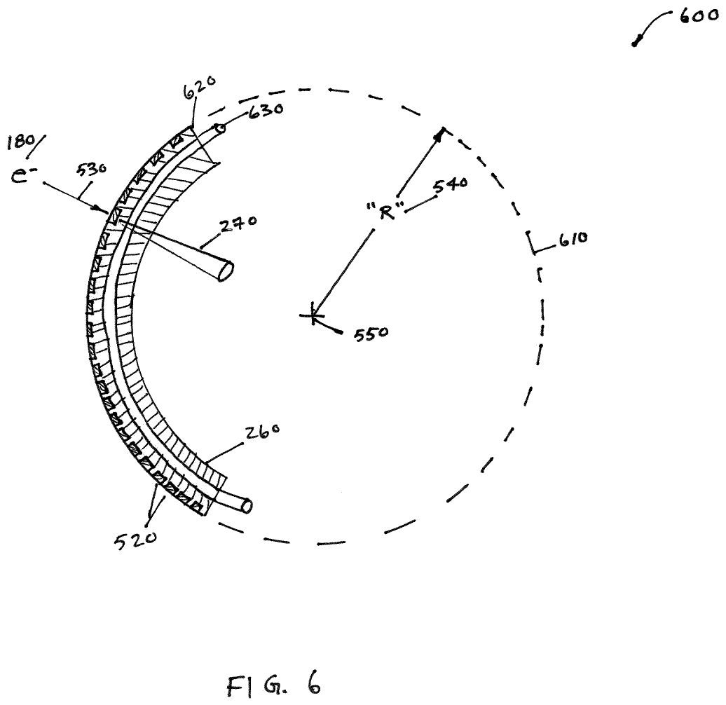

Referring now to FIG. 6, a cross-sectional view 600 of an embodiment of the target assembly 260 is shown, having separate individual targets 520, with a striking surface that is oriented substantially perpendicular to the trajectory 530 of the incoming electron beam 180. The targets 520 may be positioned on a circular arc 610, all having substantially the same distance or focal length or radius R and having the same focal point 550. The targets 520 may be brazed or metallurgically bonded at high temperature (e.g., greater than about 800° C.) to a suitable heatsink 620 made from copper or another vacuum comparable material with high thermal conductivity. The pulsed electrons 180 impacting the targets 520 can tunnel through the target material into the heatsink 620, such that the heatsink 620 is also a Bremsstrahlung converter where photons 270 are created. The heatsink 620 is in contact with coolant 630, such as water, which flows to and from a heat exchanger or cooling unit.

While it is preferable for the coolant to be positioned away from the path of the photons 270, the cooling may be more effective when the coolant is in contact with the copper surface that is on-axis with the pulsed electron beam 180 and subsequent photons 270.

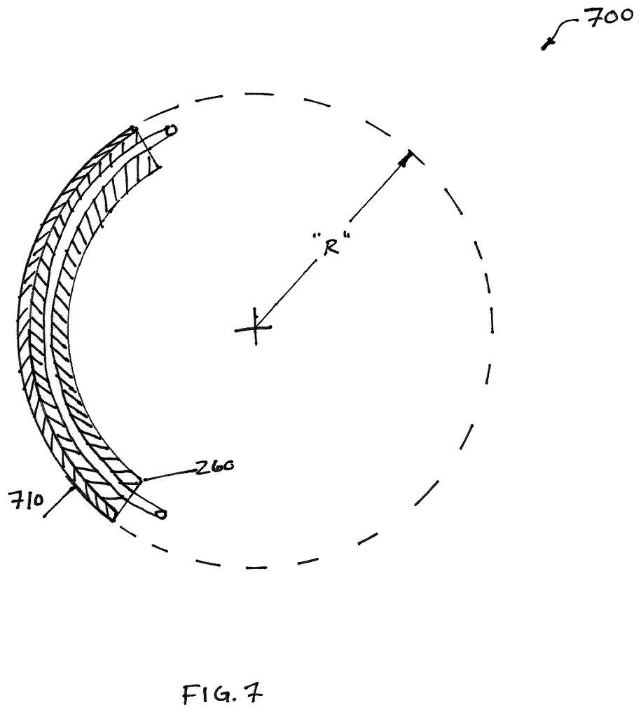

Referring now to FIG. 7, a cross-sectional view 700 is shown of an alternative embodiment to the view 600 using a single continuous target 710 instead of the individual targets 520. The continuous target 710 is impacted by the pulsed electron beam 180 in a series of different locations, similar to how the target assembly 260 uses multiple distinct targets 520 to spread the heat over a large enough surface area to prevent thermal failure. The coolant path is shown to be in-line with the path of the photon beams 270, but it should be understood that the coolant may instead take a different path.

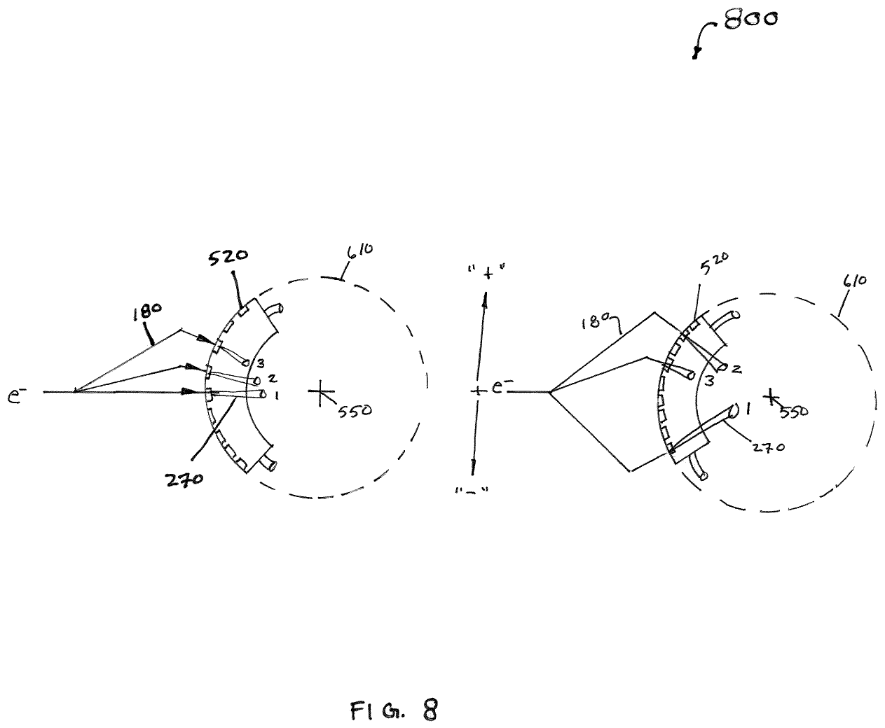

Referring now to FIG. 8, a schematic 800 shows how adjacent targets 520, positioned on an arc 610, can be impacted by the pulsed electron beam 180 in different sequences. For example, a set of three beams are shown, numbered in order of execution. In the example on the left-hand side, the three beams correspond to adjacent targets 520. In the example on the right-hand side, the three beams correspond to targets 520 that are at arbitrary points along the arc 610. The electron beam 180 may be directed at targets 520 in any appropriate order to effect the radiotherapy treatment while keeping the target temperature below a predetermined temperature threshold. The electron beams 180 may further be aimed in the “+” region above the Z-axis and the “−” region below the Z-axis to create photon beams 270 from different angles, having axes that converge at a common focal point 550.

The number of individual targets 520, or the number of distinct impact locations on a continuous target 710, may be determined by the operating parameters and the maximum allowable energy in a single pulse that the target material can withstand. The higher the number of targets 520, or the more impact locations on a continuous target 710, the greater the number of angles that can be used to treat the lesion.

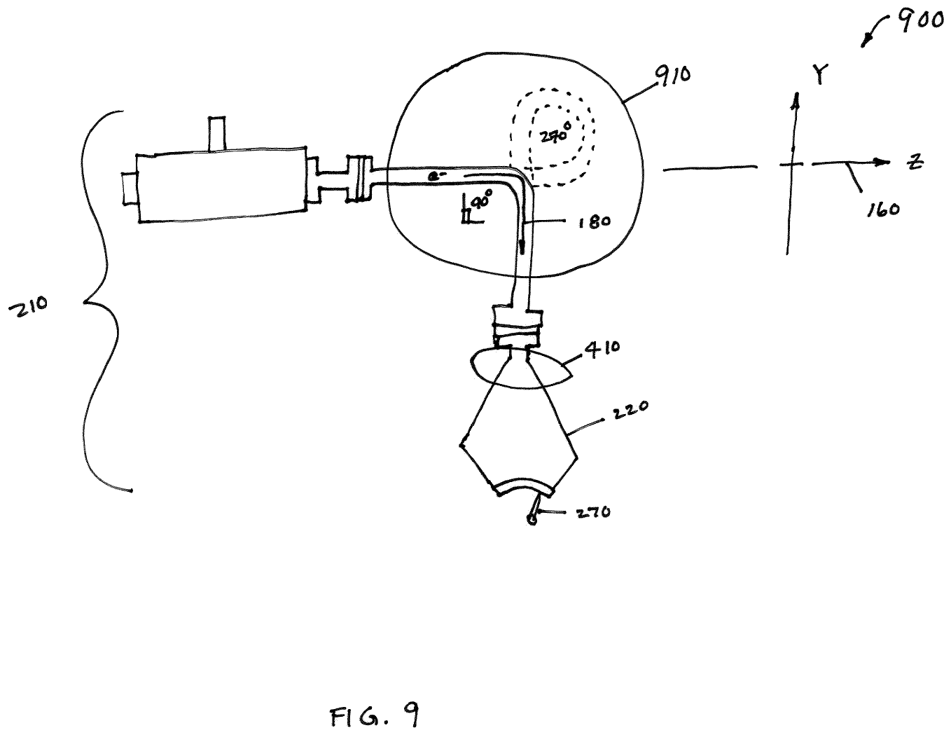

Referring now to FIG. 9, an alternative embodiment 900 to the schematic 200 is shown. A redirecting magnetic field 910 or a series of magnetic fields bend the electron beam 180 by 90° or by 270° into a direction along the Y-axis before the scan section 220, where the scan magnetic field 410 deflects the beam off its new Y-axis trajectory. In this configuration, the patient may be positioned horizontally, perpendicular to the incoming photon beams 270 and can be treated in the transverse plane 310 or sagittal plane 215, depending on the rotational orientation of the beam line 210.

The first pulsed electron beam 180 may have a trajectory 530 on the Z-axis or on the same axis as the linear electron accelerator 110, or can start at any of the possible alternative trajectories 530. The pulsed electron beam 180 dwells briefly on the target 520 and begins delivering its treatment dose. Before the next pulse begins, as determined by the pulse repetition rate, the scan magnetic field 410 is altered to aim the electron beam 180 to the next target 520 or the next location on continuous target 710. This keeps each impact location at a low enough temperature to prevent thermal failure, while producing radiotherapy level dose rates from each target impact location.

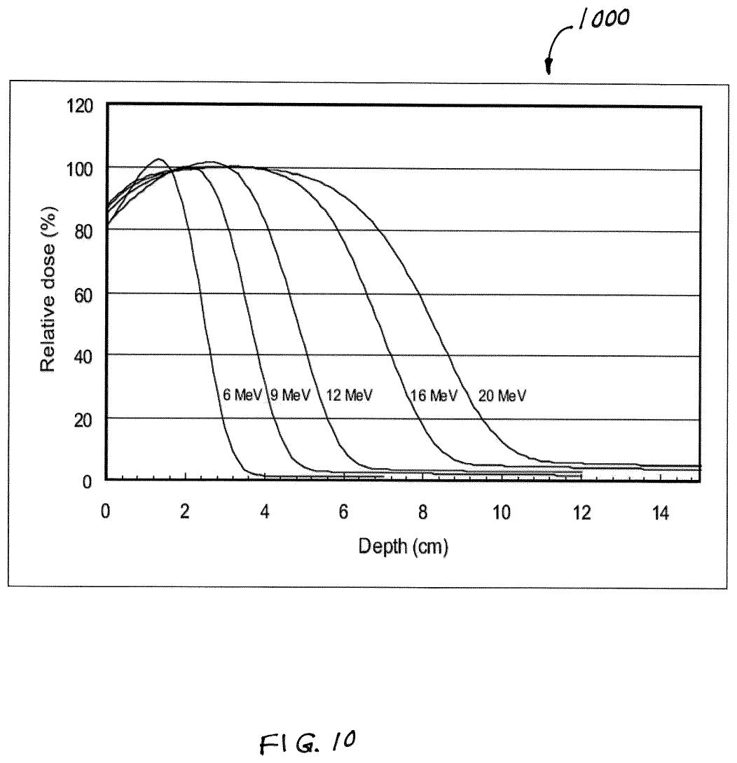

Referring now to FIG. 10, a graph 1000 is shown of a qualitative depth dose curve for electron beams traveling through water, having different energies. The horizontal axis indicates a depth in centimeters, for example in a person's body, while the vertical axis indicates a relative dose produced by the electrons as a percentage of the maximum dose. The depth dose curves for photons in water have a very similar attenuation behavior, and their behavior in a human body is very similar their attenuation in water. The graph 1000 indicates how the beams are attenuated as they travel through tissue. It should be understood that this graph is intended to illustrate the relationship between depth and dosage in the body of a patient, but is not intended to precisely reflect specific quantities.

As the photons enter the body of a patient, they interact with the patient's tissue, for example freeing electrons that, in turn, progress further into the patient's body. This results in a compounding effect near the surface of the tissue, as these freed electrons add to the dose provided by the photons. Thus the maximum dose for megavolt beams tends to be delivered below the surface of the tissue, after which the relative dose starts to fall off as the electron or photon beam becomes attenuated.

By selecting the energy of the electron beam and subsequently the energy of the photon beam, the depth of the tissue receiving the maximum dose can be controlled. The energy of the photons in the photon beam correspond to the energy of the electron beam, and is a quantity distinct from the intensity of the photon beam. The intensity of the photon beam—the number of photons that reach the tissue over a unit area and unit time—is determined by the current and the energy level of the electron beam. Thus depth of the maximum dose can be controlled by the energy of the electron beam (e.g., determined by the accelerating voltage in the linear electron accelerator 110), while the magnitude of that maximum dose can be controlled by the current of the electron beam.

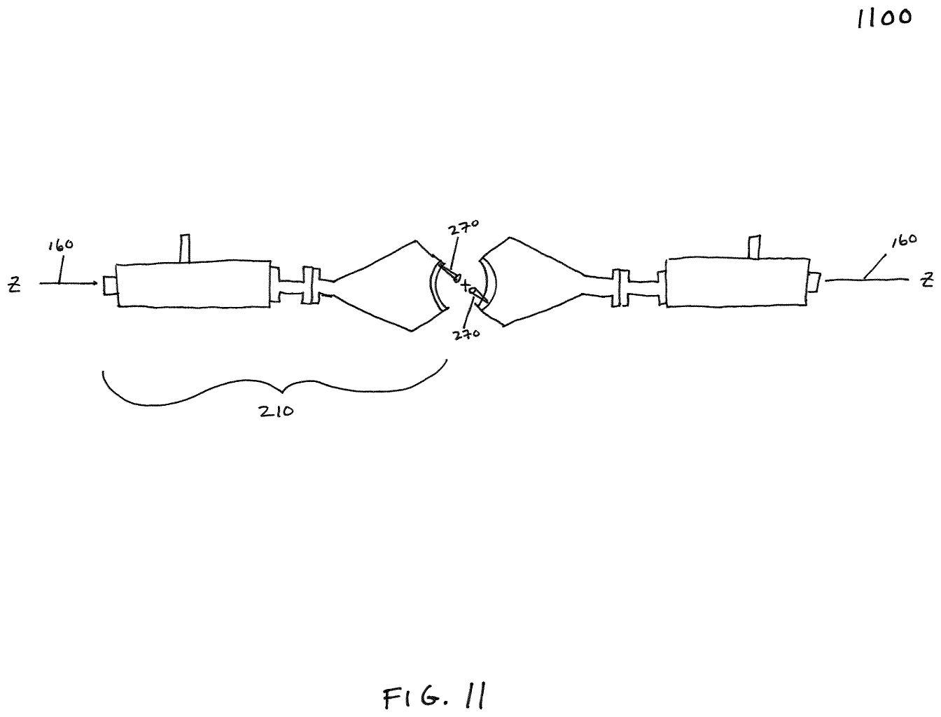

Referring now to FIG. 11, a schematic 1100 is shown of two separate beam lines 210, each with respective magnetic fields and target assemblies that are set in substantially equal and opposing locations relative to the patient. The pulsed electron beams 180 generated by the respective beam lines 210 and the subsequent photon beams 270 are shown as being on the same Z-axis 160 and are directed to a same spot, such that the dose rates for each beam line 210 combine additively. This produces double the dose rate at the focal point 550, making it easier to achieve the desired radiotherapy dose rate. As shown in this diagram, the electron beams 180 may be directed from opposite directions to intersect a same focal region.

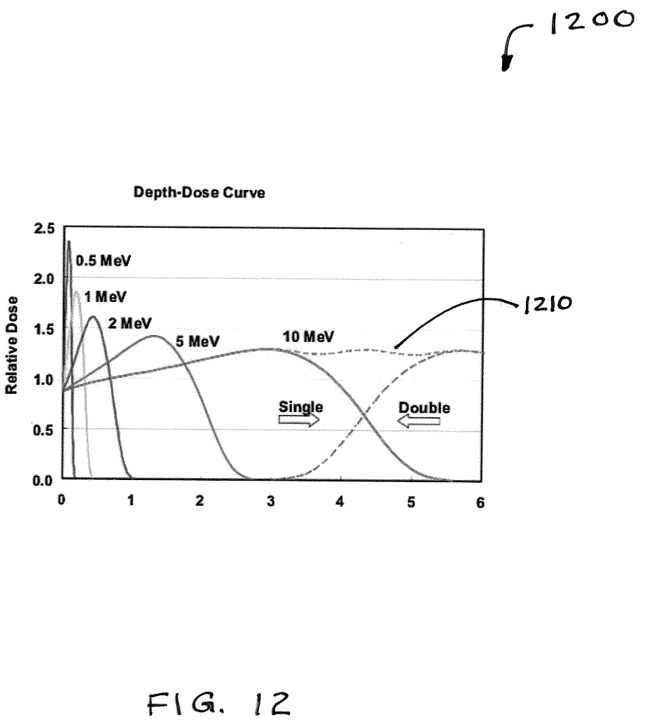

Referring now to FIG. 12, a graph 1200 is shown that illustrates how the dose depth curves for different beams compare to one another and how two 10 MV beams can combine. The horizontal axis shows the depth in centimeters, for example in a person's body, while the vertical axis shows a relative dose. A first 10 MV beam is shown with a solid line and its direction of propagation through the tissue is labeled as “Single,” while a second 10 MV beam is shown with a dashed line and its direction of propagation through the tissue is labeled as “Double.” The combined electron dose in the example shown in 1210 is the combination of the dose levels of the two 10 MV beams, reaching a higher dose at a depth (e.g., about 4.5 cm) than would be possible with one beam alone. The same holds true for the dose delivered by photon beams.

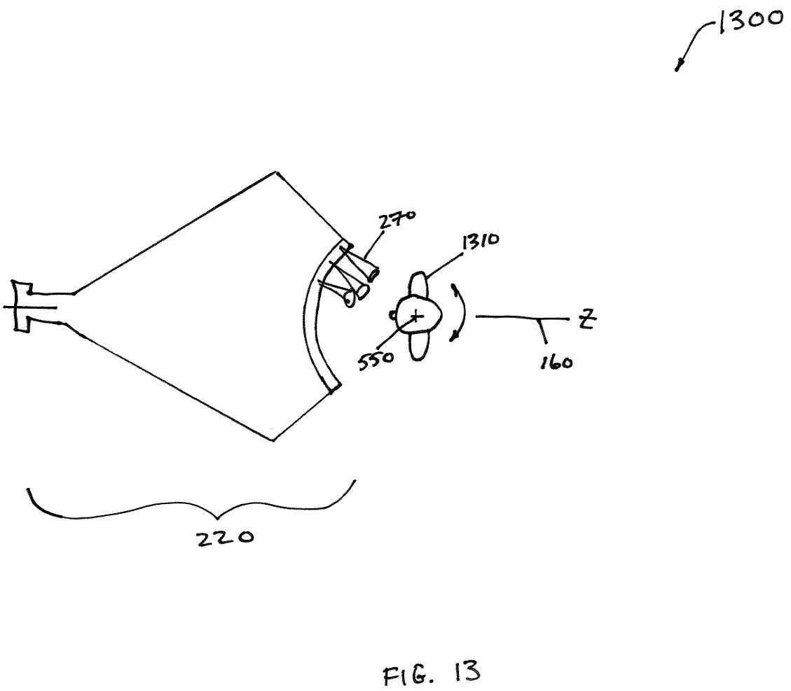

Referring now to FIG. 13, a schematic 1300 is shown of the scan section 220 in a horizontal plane, viewed from above with respect to a patient 1310. The patient 1310 may be rotated, in either or both directions, allowing the photon beams 270 to treat portions of the patient's lesion that cannot be directly targeted when the patient is stationary or to treat the patient's lesion from angles that cannot be directly achieved when the patient is stationary.

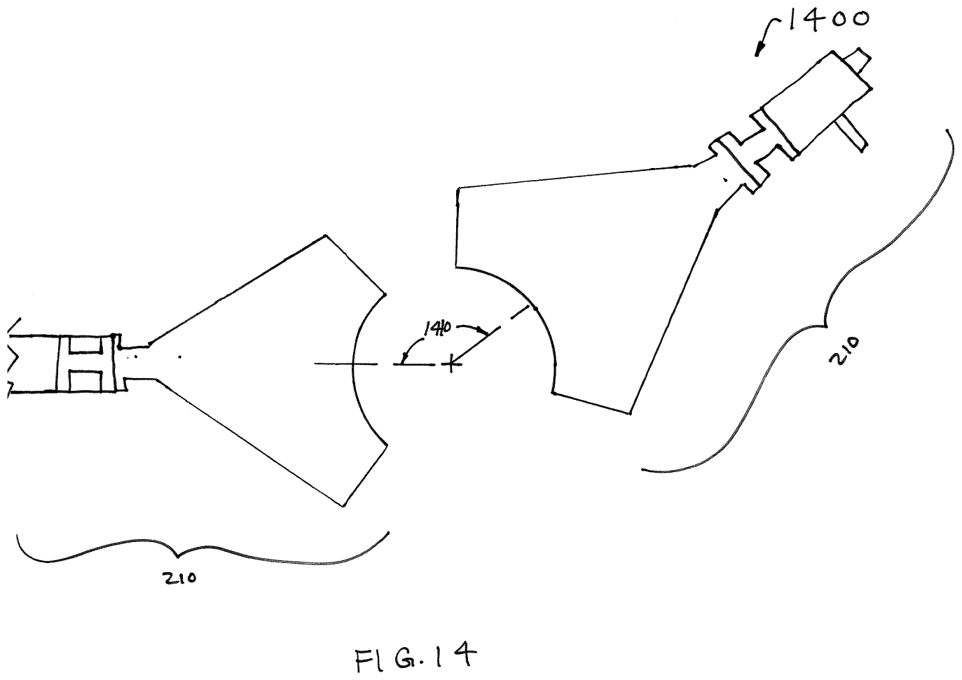

Referring now to FIG. 14, two beam lines 210 are shown being positioned in different axes from one another. These axes can form any angle 1410 with respect to one another, and may be non-coplanar. Positioning the beam lines 210 in this manner can help to minimize the dose received by healthy tissues in a patient's body, while maintaining the dose rate in a target region.

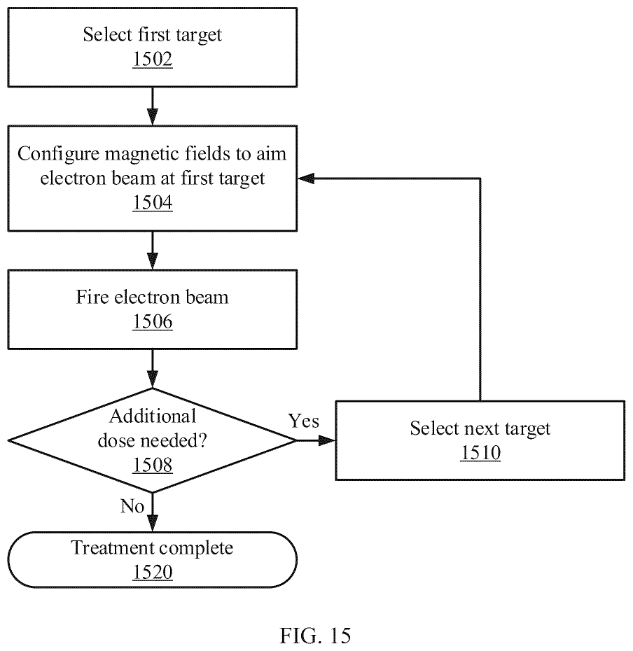

Referring now to FIG. 15, a method of performing radiotherapy is shown using a system as described above. Block 1502 selects a first target, for example from a set of independent targets 520 or as a location on a continuous target 710. Block 1504 then configures the scan magnetic fields 410 to cause an electron beam to diverge from the Z-axis and then to converge back to the Z-axis, with a trajectory that impacts the selected target. When block 1506 fires the electron beam 180, for example using linear electron accelerator 110 to generate and accelerate the beam of electrons 180 to a target energy, the converging trajectory of the electron beam 180 is directed toward the lesion 280 in a patient's body, delivering a dose of ionizing radiation thereto. The electron beam 180 is fired for a period of time that is determined by the thermal limits of the target. In particular, the period of time is selected to prevent thermal damage to the target.

Block 1508 determines whether the dose that has been administered to the lesion 280 is sufficient. If additional dose is needed, block 1510 selects a next target 520 (or a next location on continuous target 710) and processing returns to block 1504. Once a sufficient dose has been administered, processing ends.

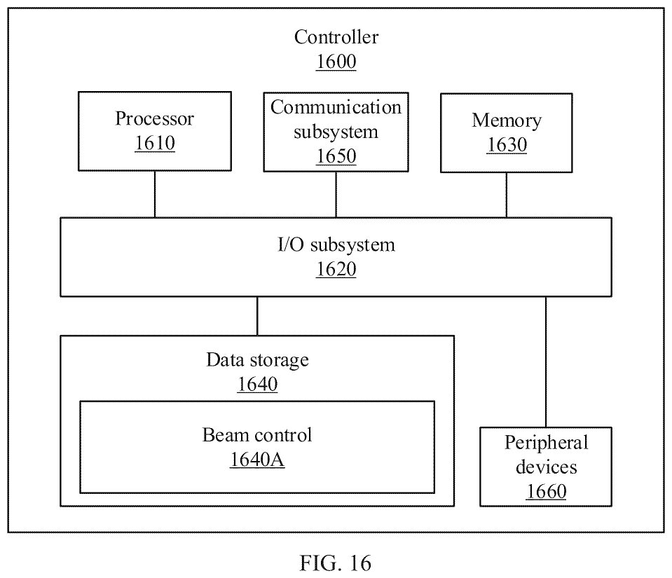

Referring now to FIG. 16, a diagram of a controller 1600 is shown. The controller 1600 is configured to perform skill imitation. The computing device controller 1600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 1600 may be embodied as one or more compute sleds, memory sleds, or other racks, sleds, computing chassis, or other components of a physically disaggregated computing device. In some embodiments, the computing device 1600 may be an application-specific integrated circuit, single-board computer, or field programmable gate array.

As shown in FIG. 16, the controller 1600 illustratively includes the processor 1610, an input/output subsystem 1620, a memory 1630, a data storage device 1640, and a communication subsystem 1650, and/or other components and devices commonly found in a server or similar computing device. The computing device 1600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 1630, or portions thereof, may be incorporated in the processor 1610 in some embodiments.

The processor 1610 may be embodied as any type of processor capable of performing the functions described herein. The processor 1610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 1630 may be embodied as any type of volatile or non-volatile memory or data storage capable of performing the functions described herein. In operation, the memory 1630 may store various data and software used during operation of the computing device 1600, such as operating systems, applications, programs, libraries, and drivers. The memory 1630 is communicatively coupled to the processor 1610 via the I/O subsystem 1620, which may be embodied as circuitry and/or components to facilitate input/output operations with the processor 1610, the memory 1630, and other components of the computing device 1600. For example, the I/O subsystem 1620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 1620 may form a portion of a system-on-a-chip (SOC) and be incorporated, along with the processor 1610, the memory 1630, and other components of the computing device 1600, on a single integrated circuit chip.

The data storage device 1640 may be embodied as any type of device or devices configured for short-term or long-term storage of data such as, for example, memory devices and circuits, memory cards, hard disk drives, solid state drives, or other data storage devices. The data storage device 1640 can store program code 1640A controlling the angle of an electron beam. For example, beam control 1640A may set a current that is applied to an electromagnetic in a controllable field generator. Any or all of these program code blocks may be included in a given computing system. The communication subsystem 1650 of the computing device 1600 may be embodied as any network interface controller or other communication circuit, device, or collection thereof, capable of enabling communications between the computing device 1600 and other remote devices over a network. The communication subsystem 1650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 1600 may also include one or more peripheral devices 1660. The peripheral devices 1660 may include any number of additional input/output devices, interface devices, and/or other peripheral devices. For example, in some embodiments, the peripheral devices 1660 may include a display, touch screen, graphics circuitry, keyboard, mouse, speaker system, microphone, network interface, and/or other input/output devices, interface devices, and/or peripheral devices.

Of course, the computing device 1600 may also include other elements (not shown), as readily contemplated by one of skill in the art, as well as omit certain elements. For example, various other sensors, input devices, and/or output devices can be included in computing device 1600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 1600 are readily contemplated by one of ordinary skill in the art given the teachings of the present invention provided herein.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that those skilled in the art may implement various modifications without departing from the scope and spirit of the invention. Those skilled in the art could implement various other feature combinations without departing from the scope and spirit of the invention.

1. A vacuum electron device (VED), comprising:
an electron accelerator that generates an electron beam from an electron source along a central axis;
a plurality of targets, arranged on a circular arc, that generate photons upon impact by the electron beam; and
a magnetic controllable field generator that generates a magnetic field to change a trajectory of the electron beam, including a first field region that causes the electron beam to diverge from the central axis and a second field region that causes the electron beam to converge toward the central axis to impact a selected target of the plurality of targets.

2. The VED of claim 1, wherein the plurality of targets include a plurality of respective pieces of target material that are separated from one another on a target assembly.

3. The VED of claim 1, wherein the plurality of targets include different respective locations on a continuous structure of target material.

4. The VED of claim 1, further comprising a collimator having a plurality of apertures, each aperture corresponding to a respective target of the plurality of targets.

5. The VED of claim 4, wherein the plurality of apertures are arranged on a circular arc that has a same center point as the circular arc of the plurality of the targets.

6. The VED of claim 4, wherein the plurality of apertures each have a shape selected from the group consisting of a cone, a cylinder, and an asymmetrical shape tailored to a lesion.

7. The VED of claim 1, wherein the circular arc has a center point that corresponds to a convergence of the trajectory of the electron beam and subsequently the photon beams and the central axis for all of the plurality of targets.

8. The VED of claim 1, further comprising a controller that operates the controllable magnetic field generator to change an impact location between targets of the plurality of targets.

9. A vacuum electron device (VED), comprising:
an electron accelerator that generates an electron beam from an electron source along a first axis;
a redirecting magnetic field that redirects the electron beam from the first axis to a second axis;
a plurality of targets, arranged on a circular arc, that generate photons upon impact by the electron beam; and
a controllable field generator that generates a magnetic field to change a trajectory of the electron beam, including a first field region that causes the electron beam to diverge from the second axis and a second field region that causes the electron beam to converge toward the second axis to impact a selected target of the plurality of targets.

10. The VED of claim 9, wherein the second axis is perpendicular to the first axis.

11. The VED of claim 9, wherein the plurality of targets include a plurality of respective pieces of target material that are separated from one another on a target assembly.

12. The VED of claim 9, wherein the plurality of targets include different respective locations on a continuous structure of target material.

13. The VED of claim 9, further comprising a collimator having a plurality of apertures, each aperture corresponding to a respective target of the plurality of targets.

14. The VED of claim 9, wherein the circular arc has a center point that corresponds to a convergence of the trajectory of the electron beam and the second axis for all of the plurality of targets.

15. The VED of claim 9, further comprising a controller that operates the controllable magnetic field generator to change between targets of the plurality of targets.

16. A vacuum electron device (VED), comprising:
a first electron accelerator that generates a first electron beam from a first electron source along a central axis in a first direction;
a second electron accelerator that generates a second electron beam from a second electron source along the central axis in a second direction, opposite to the first direction;
a first plurality of targets, arranged on a first circular arc, that generate photons upon impact by the first electron beam;
a second plurality of targets, arranged on a second circular arc, that generate photons upon impact by the second electron beam;
a first controllable magnetic field generator that generates a first magnetic field to change a trajectory of the first electron beam, including a first diverging region that causes the first electron beam to diverge from the central axis and a first converging field region that causes the first electron beam to converge toward the central axis to impact a selected first target of the first plurality of targets; and
a second controllable magnetic field generator that generates a second magnetic field to change a trajectory of the second electron beam, including a second diverging region that causes the second electron beam to diverge from the central axis and a second converging field region that causes the second electron beam to converge toward the central axis to impact a selected second target of the second plurality of targets,
wherein photons generated by the first target and photons generated by the second target intersect a same focal region.

17. The VED of claim 16, wherein the first plurality of targets include a plurality of respective pieces of target material that are separated from one another on a target assembly.

18. The VED of claim 16, wherein the first plurality of targets includes different respective locations on a continuous structure of target material.

19. The VED of claim 16, further comprising a first collimator having a plurality of apertures, each aperture corresponding to a respective target of the first plurality of targets.

20. The VED of claim 16, wherein the first circular arc and the second circular arc have a same center point at the focal region that corresponds to a convergence of the trajectory of the first electron beam, the trajectory of the second electron beam and the central axis for all of the plurality of targets.
